(19) United States
(12) Patent Application Publication
(10) Pub. No.: US 2011/0305744 A1
(43) Pub. Date: Dec. 15, 2011

(54) PREPARATION FOR VAGINAL AND RECTAL USE AND RELATIVE PRODUCTION METHOD

(75) Inventor: Vincenzo Russo, Bologna (IT)

(73) Assignee: FARMA-DERMA SRL, Sala Bolognese (BO) (IT)

(21) Appl. No.: 12/877,475

(22) Filed: Sep. 8, 2010

(30) Foreign Application Priority Data
Jun. 9, 2010 (IT) UD2010A000112

(51) Int. Cl. A61K 36/82; A61K 9/02; A61K 31/728; A61P 43/00; C07H 5/06; A61K 36/00; A61K 36/736; A61P 15/02; A61K 9/00; A61K 36/886

(52) U.S. Cl. 424/430; 424/436; 514/54; 424/744; 536/55.1; 424/725; 424/729; 424/735

(57) ABSTRACT

A preparation for vaginal and rectal use comprising hyaluronic acid, with an average particle size comprised between about 50 micrometers and about 200 micrometers and a molecular weight comprised between about 1,000,000 Da and about 1,800,000 Da.

### FIELD OF THE INVENTION

The present invention concerns a preparation for vaginal and rectal use, its composition and the relative production method. In particular the preparation of the present invention can be used in repairing the vaginal and rectal mucosa, for example, but not only, in the form of a vaginal pessary or rectal suppository.

### BACKGROUND OF THE INVENTION

It is known from the European patent EP-B-1.275.395 in the name of the present Applicant to produce a preparation in the form of vaginal pessaries formed from semi-synthetic glycerides, suitable to be introduced into the vagina and which dissolve and can be easily eliminated from the body. Hyaluronic acid (HA) and possibly other active principles are incorporated into the semi-synthetic glycerides. The vaginal pessaries are normally used for the treatment of cervicovaginal atrophy and dystrophic conditions, delay in the process of vulvo-vaginal tissue repair after childbirth, in gynecological surgery, in dystrophies that follow chemotherapy treatments or based on ionizing radiations.

Hyaluronic acid is a natural compound of soft biological tissues, widely distributed in the human body. It is mainly found in the skin and in the connective tissue, where it is synthesized by fibroblasts.

Hyaluronic acid is an element of the glycosaminoglycan family formed by a regular repetitive sequence of the non-sulphated disaccharide unit formed by glucuronic acid and N-acetylglucosamine [Beta D-glucoronic acid 1,3-N-acetyl-beta-D-glucosamine, 4]n.

Carboxyl groups of glucuronic acid and also those of N-acetylglucosamine, in vivo, are completely ionized, giving the molecule a high degree of polarity and, consequently, a greater solubility in water.

Hyaluronic acid can reach a level of mobilization in water up to 20 times its own weight.

It has a very high molecular mass, its molecular weight varies from 4,000 Da to 8,000,000 Da and it has interesting hydrophilic, rheological and viscoelastic properties, based on its polymeric and polyelectrolyte characteristics. Moreover, despite the simple primary structure, hyaluronic acid molecules of different molecular weight have cell signaling functions that are extraordinarily variable and often opposite. Extracellular polymers of a high molecular weight have the capacity of spatial refill, hydration and also an anti-angiogenic and immunosuppressive effect; on the contrary polymers with a low molecular weight have a pro-angiogenic action, immunostimulating and inflammatory. The process of micronization is known to reduce the average diameter of the particles of a solid material.

Traditional micronization techniques are based on friction in order to reduce the size of the particles. Such methods include milling, grinding and sieving, as is described in application WO-A-0243701.

Modern methods use the supercritical fluids (SCFs) in the micronization process. These techniques are, for example, the rapid expansion of supercritical solutions (RESS), supercritical crystallization with anti-solvents (SAS) and production of particles from saturated gas solutions (PGSS).

Micronization techniques based on supercritical fluids are promising for the production of particles with controlled sizes and distribution. In pharmaceutical industries, many medicines, especially the newly developed substances, are not very water-soluble, which limits their theoretical oral bioavailability, and micronization can be used to increase their absorption because the dissolution speed is raised by reducing the size of the particles.

Moreover, micronization can be used to improve the mixing efficiency. The ingredients have to be accurately and rigorously mixed in order to ensure a uniform microstructure over all of the desired pharmaceutical form. An inadequate mix of the ingredients and the formation of defects causes a lack of homogeneity, such as agglomerates, which seriously reduce the performance of the product. The degree of mixing depends on various factors, the first of which is the form and size of each single component.

Micronization can be used to raise the quality of the mix since the degree of mixing is improved by the reduction of the size of the particles.

Purpose of the present invention is to make a preparation for vaginal and rectal use, and to perfect a relative preparation method, which creates optimal conditions, in terms of humid environment and physical barrier, for the treatment in tissue repair of the vaginal and rectal mucosa deriving from damage of any origin and nature.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purpose, a preparation for vaginal and rectal use according to the present invention comprises hyaluronic acid.

According to a characteristic feature of the present invention, the hyaluronic acid is micronized with an average particle size comprised between about 50 micrometers and about 200 micrometers.

In some forms of embodiment, the hyaluronic acid has an average particle size comprised between about 50 micrometers and about 180 micrometers.

According to another feature of the present invention, the hyaluronic acid has a molecular weight comprised between about 1,000,000 Da and about 1,800,000 Da (hyaluronic acid at a medium molecular weight-MMW).

In some forms of embodiment the hyaluronic acid has a molecular weight comprised between about 1,450,000 Da and about 1,770,000 Da.

In accordance with some forms of embodiment of the present invention, the micronized hyaluronic acid MMW is incorporated in a lipophilic excipient. The lipophilic excipient is advantageous in determining the structure of the preparation of the present invention, for example in the form of vaginal pessaries or rectal suppositories.

In some forms of embodiment, the micronized hyaluronic acid derives from a micronizing process which uses supercritical fluids (SCFs). Alternatively it is possible to use traditional micronizing techniques, such as milling, grinding or sieving, as described for example in application WO-A-0243701.

The micronized nature of the hyaluronic acid with average sizes between about 50 micrometers and about 200 micrometers allows to obtain a homogenous distribution of the hyaluronic acid inside the mass of the lipophilic excipient and to prevent the formation of agglomerates during the stirring and mixing process of the components of the preparation, which is typically done in a thermostatic melting device.

Different molecular weights of the hyaluronic acid show different mechanical and hydration effects: hyaluronic acid with a low molecular weight penetrates deeply into the tissue and provides a deeper hydration, while hyaluronic acid with a medium to high molecular weight forms a protective film and prevents dehydration.

The range of molecular weight according to the present invention between about 1,000,000 Da and about 1,800,000 Da allows to ensure the better formation of film and protective barrier effect, with an optimal compromise between mechanical properties and hydration properties.

The combination of micronized hyaluronic acid with medium size particles comprised between about 50 micrometers and about 200 micrometers and selected molecular weight between about 1,000,000 Da and about 1,800,000 Da allows to create optimal conditions, in terms of environment humidity and physical barrier, for the treatment of tissue repair of the vaginal and rectal mucosa deriving from damage of any origin or nature.

In some forms of embodiment, the hyaluronic acid can be combined, in the preparation according to the present invention, with one or more other active substances selected from a group containing Aloe Vera, Centella Asiatica, Calendula and Tea Tree oil. Aloe Vera and Centella Asiatica, added to the basic hyaluronic acid, give the preparation cicatrizant and anti-inflammatory properties. Moreover, Calendula has an antiedemigene action and Tea Tree oil has a mild antibacterial action.

In some forms of embodiment, sweet almond oil can be added, which has soothing and emollient properties.

As a whole the preparation has a beneficial and salutary effect on the vaginal and rectal mucosa.

In some forms of embodiment, the active substances of the present invention, in particular hyaluronic acid, are incorporated into a pessary or suppository provided for the purpose and made to a large extent based on said lipophilic excipient.

The lipophilic excipient is advantageous for the incorporation of hyaluronic acid, which instead has a hydrophilic nature. The chosen lipophilic excipient has the property that it melts quickly so that it can be easily eliminated from the body. A lipophilic excipient which is commonly used is semi-synthetic glycerides which have these advantageous properties.

Semi-synthetic glycerides are a mass of triglycerides of hydrogenated fatty acids deriving from palm oil and palm heart oil, with the shape and color of a waxy white pellet and a melting point of between about 34° C. and 36° C.

Thanks to the hydrophilic nature of the hyaluronic acid, in contrast to the lipophilic nature of the excipient, there is a better capacity of releasing the active substances. When the lipophilic excipient melts and is eliminated, the active substances, in particular the hyaluronic acid, stay in situ to interact with the mucosa.

In some forms of embodiment, the preparation according to the present invention is in the form of a vaginal pessary formed by said lipophilic excipient, suitable to be introduced into the vagina for use in treatment of tissue repair of the vaginal mucosa.

In other forms of embodiment, the preparation according to the present invention is in the form of a rectal suppository formed by said lipophilic excipient, suitable to be introduced into the rectal canal for use in treatment of tissue repair of the anorectal mucosa.

In some forms of embodiment, for the production of the preparation according to the present invention a quantity of hyaluronic acid is used with a percentage in weight, with respect to the overall preparation, comprised between about 0.1% and about 1%.

In some forms of embodiment of a pessary or suppository with a weight comprised between about 2 g and about 5 g, a quantity of hyaluronic acid comprised between about 2 mg and about 20 mg is used.

In some forms of embodiment, for the production of the preparation according to the present invention, a quantity of Aloe Vera is used with a percentage in weight, with respect to the overall preparation, comprised between about 1% and about 5%.

In forms of embodiment of a pessary or suppository with a weight comprised between about 2 g and about 5 g, a quantity of Aloe Vera comprised between about 20 mg and about 100 mg is used.

In some forms of embodiment, for the production of the preparation according to the present invention a quantity of Centella Asiatica is used with a percentage in weight, with respect to the overall preparation, comprised between about 1% and about 5%.

In forms of embodiment of a pessary or suppository with a weight comprised between about 2 g and about 5 g, a quantity of Centella Asiatica comprised between about 20 mg and about 100 mg is used.

In some forms of embodiment, for the production of the preparation according to the present invention a quantity of Calendula is used with a percentage in weight, with respect to the overall preparation, comprised between about 1% and about 5%.

In forms of embodiment of a pessary or suppository with a weight comprised between about 2 g and about 5 g, a quantity of Calendula comprised between about 20 mg and about 100 mg is used.

In some forms of embodiment, for the production of the preparation according to the present invention a quantity of Tea Tree oil is used with a percentage in weight, with respect to the overall preparation, comprised between about 0.05% and about 0.5%.

In forms of embodiment of a pessary or suppository with a weight comprised between about 2 g and about 5 g, a quantity of Tea Tree oil comprised between about 1 mg and about 10 mg is used.

In some forms of embodiment, for the production of the preparation according to the present invention a quantity of sweet Almond oil is used with a percentage in weight, with respect to the overall preparation, comprised between about 1% and about 5%.

In forms of embodiment of a pessary or suppository with a weight comprised between about 2 g and about 5 g, a quantity of Tea Tree oil comprised between about 20 mg and about 100 mg is used.

The form of embodiment and application by means of pessaries or suppositories is particularly advantageous because it allows the active substances, in particular hyaluronic acid, to be kept in the vagina or in the rectal canal for a long period and released gradually.

The lipophilic excipients are useful because they give to the pessary or suppository, the right consistency and influence positively the release of the active substances, in particular the hyaluronic acid.

A preparation as described above also comes within the scope of the present invention, for use in the treatment of tissue repair of the vaginal mucosa, cervicovaginal atrophy and relative dystrophic conditions (vaginitis due to a lack of estrogen, senile vaginitis, vulva-vaginal craurosis), delay in the process of vulvo-vaginal tissue repair after childbirth, in gynecological surgery, in dystrophies that follow chemotherapy treatments or treatments based on ionizing radiations and also in use for auxiliary treatment of the reparatory processes of the anorectal mucosa after proctological surgery, internal and external hemorrhoids, proctitis, cryptitis, anal rhagades, perianal fissures and fistulae.

The present invention also concerns a method the production of a preparation for vaginal and rectal use comprising hyaluronic acid, which uses micronized hyaluronic acid with an average particle size comprised between about 50 micrometers and about 200 micrometers and with a molecular weight comprised between about 1,000,000 Da and about 1,800,000 Da.

In some forms of embodiment of the present invention the hyaluronic acid is incorporated into a lipophilic excipient. In some forms of embodiment of the present invention the lipophilic excipient is melted and mixed and at least the hyaluronic acid is subsequently added. In some forms of embodiment of the present invention, other active substances are also added, such as Aloe Vera and/or Centella Asiatica and/or Calendula and/or Tea Tree Oil and/or sweet Almond Oil. In some forms of embodiment of the present invention the hyaluronic acid is added after the other active substances. In some forms of embodiment of the present invention the hyaluronic acid and the possible other active substances are added after a desired period of time has passed from the melting of the lipophilic excipient. In some forms of embodiment of the present invention, after having added the hyaluronic acid and the possible other active substances, the method provides to let a further period of time pass, during which the melted mass is kept stirred. In some forms of embodiment, at the end of this period of time, the cells of a preformed blister are filled, for example having a shape mating with that of a pessary or a suppository, with the melted mass which contains the hyaluronic acid and the possible other active substances.

The use of micronized hyaluronic acid as described above has advantages in the production step in terms of reduction of times, of a mechanical type, in the precision of dosage as well as in the quality of mixing.

In particular, in the state of the art where non-micronized hyaluronic acid is used, in the pre-mixing step the agglomerates in the non-micronized hyaluronic acid, if present, have to be reduced manually, before it is inserted into the melted mass of the excipient, and in the mixing step the presence of visible agglomerates in the melting device entails prolonging the stirring time, whereas with the use of micronized hyaluronic acid according to the present invention this waste of time can be avoided.

The advantages of a mechanical type are that particles of hyaluronic acid do not deposit on the mechanical parts of the machines used, in particular preventing the blockage of the nozzle which dispenses the melted mass into the single cells, thus giving a significant reduction in the frequency of cleaning interventions.

The precision of the dosage is given by the fact that the reduced dispersion, as a result of using micronized hyaluronic acid, allows a greater precision in the dosing/filling step of the melted mass into the cells.

Finally, since the micronized hyaluronic acid is distributed uniformly inside the melted mass, it is possible to reduce the speed of the rotor which mixes the melted mass, obtaining gentle mixing conditions which are more convenient.

### DESCRIPTION OF SOME FORMS OF EMBODIMENT

Forms of embodiment of the present invention relate to a pessary or suppository formed for the most part by semi-synthetic glycerides, into which micronized hyaluronic acid is incorporated with average size particles comprised between about 50 micrometers and about 200 micrometers, in some forms of embodiment between about 50 micrometers and about 180 micrometers and with a molecular weight comprised between about 1,000,000 Da and about 1,800,000 Da (hyaluronic acid MMW), in some forms of embodiment between about 1,450,000 Da and about 1,770,000 Da, for example about 1,600,000 Da.

In some forms of embodiment the hyaluronic acid is micronized, or bought commercially already in this form.

In some forms of embodiment, the micronized hyaluronic acid is obtained by means of a traditional micronizing process by means of milling, grinding or sieving, as described in the application WO-A-0243701.

In other forms of embodiment, the micronized hyaluronic acid is obtained by means of a micronizing process which uses supercritical fluids, with the advantage of having an optimal control of the average size, an increase in the absorption capacity given by the increased dissolution speed, which is increased in its turn by the reduction in the size of the particles. Another advantage of micronization with supercritical fluids is the improvement in the efficiency of the mixing, due to a better control of the form and size of the particles of hyaluronic acid.

In some forms of embodiment, as well as hyaluronic acid, one or more other active substances, chosen from a group comprising Aloe Vera, Centella Asiatica, Calendula, Tea Tree oil and sweet Almond oil, are incorporated into the lipophilic excipient, in this case into the semi-synthetic glycerides.

In some forms of embodiment, for the production of the preparation according to the present invention a quantity of hyaluronic acid is used with a percentage in weight comprised between about 0.1% and about 1% with respect to the overall preparation.

In some forms of embodiment, for the production of the preparation according to the present invention a quantity of Aloe Vera is used with a percentage in weight comprised between about 1% and about 5% with respect to the overall preparation.

In some forms of embodiment, for the production of the preparation according to the present invention a quantity of Centella Asiatica is used with a percentage in weight comprised between about 1% and about 5% with respect to the overall preparation.

In some forms of embodiment, for the production of the preparation according to the present invention a quantity of Calendula is used with a percentage in weight comprised between about 1% and about 5% with respect to the overall preparation.

In some forms of embodiment, for the production of the preparation according to the present invention a quantity of Tea Tree oil is used with a percentage in weight comprised between about 0.05% and about 0.5% with respect to the overall preparation.

In some forms of embodiment, for the production of the preparation according to the present invention a quantity of sweet almond oil is used with a percentage in weight comprised between about 1% and about 5% with respect to the overall preparation.

In some forms of embodiment of the production method according to the present invention, the incorporation of the hyaluronic acid is made by means of a stirring and mixing operation with the semi-synthetic glycerides in a thermostat melting device at suitable temperature. Typically, the melting temperature of the semi-synthetic glycerides occurs at a temperature between about 45° C. and about 55° C.

In particular, the semi-synthetic glycerides which determine the structural and excipient properties of the final pessary/suppository are melted in the melting device at a controlled temperature and, after a predetermined period of time, in some forms of embodiment comprised between about 25 min and about 35 min, for example about 30 min, the active substances are added, in particular the hyaluronic acid and, in some forms of embodiment, also Aloe Vera and/or Centella Asiatica and/or Calendula and/or Tea Tree Oil and/or sweet Almond Oil, to the melted mass which will form the pessaries or suppositories.

In some forms of embodiment, the hyaluronic acid according to the present invention is added as a last component to the melted mass.

In some forms of embodiment, the order in which the active substances are added to the melted mass provides to start with the possible addition of Tea Tree Oil, possibly followed by the addition of Aloe Vera, then possibly Centella Asiatica, then possibly Calendula, then possibly sweet Almond Oil and finally hyaluronic acid. In some forms of embodiment, the resulting melted mass is preferably kept at said predefined melting temperature for a further period of time, in one form of embodiment comprised between about 25 min and about 35 min, for example about 30 min. In this step of the process, the mass is kept stirred in order to proceed, after said further period of time, to fill the cells in a preformed blister having a shape mating with that of a pessary or suppository, suitable to contain a determinate quantity of said melted mass formed by the semi-synthetic glycerides, into which the active substances are incorporated.

### DESCRIPTION OF SOME PREFERENTIAL FORMS OF EMBODIMENT

For pessaries or suppositories with a weight between about 2 g and about 5 g, formed for the most part by a lipophilic excipient, for example semi-synthetic glycerides, significant results can be achieved in the tissue repair treatment of vaginal and rectal mucosa, using the following quantities of active substances added to the lipophilic excipient:
micronized hyaluronic acid MMW: from about 2 mg to about 20 mg;
Aloe Vera: from about 20 mg to about 100 mg;
Centella Asiatica: from about 20 mg to about 100 mg;
Calendula: from about 20 mg to about 100 mg;
Tea Tree Oil: from about 1 mg to about 10 mg.

The hyaluronic acid, in one form of embodiment, has average particle size between about 50 micrometers and about 180 micrometers and a molecular weight of about 1,600,000 Da.

One form of embodiment particularly preferred in the present invention provides to make a pessary/suppository which weighs about 2 g and which uses, as active ingredients, a mixture which contains the following quantities of substances:
micronized hyaluronic acid MMW as defined above: about 5 mg;
Aloe Vera: about 60 mg;
Centella Asiatica: about 60 mg;
Calendula: about 60 mg;
Tea Tree Oil: about 2 mg.

In this form of embodiment, to obtain a pessary or suppository of about 2 g, a quantity of about 1,810 g of semi-synthetic glycerides is used as lipophilic excipient.

Another example of a form of embodiment of the present invention provides to make a pessary or suppository which weighs about 2 g and which uses, as active ingredients, a mixture which contains the following quantities of substances:
micronized hyaluronic acid MMw as defined above: about 10 mg;
Sweet Almond Oil: about 80 mg.

In this form of embodiment, to obtain a pessary or suppository of about 2 g, a quantity of about 1,810 of semi-synthetic glycerides is used as lipophilic excipient.

1. A preparation for vaginal and rectal use comprising hyaluronic acid, wherein the hyaluronic acid is micronized with an average particle size comprised between about 50 micrometers and about 200 micrometers and wherein the hyaluronic acid has a molecular weight comprised between about 1,000,000 Da and about 1,800,000 Da.

2. The preparation as in claim 1, wherein the hyaluronic acid has an average particle size comprised between about 50 micrometers and about 180 micrometers.

3. The preparation as in claim 1, wherein the hyaluronic acid has a molecular weight comprised between about 1,450,000 Da and about 1,770,000 Da.

4. The preparation as in claim 1, wherein the hyaluronic acid is of the type micronized by a process with supercritical fluids, or by traditional micronization techniques such as milling, grinding or sieving.

5. The preparation as in claim 1, wherein the hyaluronic acid is incorporated in a lipophilic excipient.

6. The preparation as in claim 5, wherein said lipophilic excipient comprises semi-synthetic glycerides.

7. The preparation as in claim 5, the preparation being in the form of a vaginal pessary formed by said lipophilic excipient, suitable to be introduced into the vagina for use in treatment of tissue repair of the vaginal mucosa.

8. The preparation as in claim 5, the preparation being in the form of a rectal suppository formed by said lipophilic excipient, suitable to be introduced into the rectal canal for use in treatment of tissue repair of the anorectal mucosa.

9. The preparation as in claim 1, wherein for the production thereof a quantity of hyaluronic acid is used with a percentage in weight with respect to the overall preparation comprised between about 0.1% and about 1%.

10. The preparation as in claim 1, further comprising one or more other active substances selected from a group comprising Aloe Vera, Centella Asiatica, Calendula, Tea tree oil, sweet almond oil.

11. The preparation as in claim 10, wherein for the production thereof a quantity of Aloe Vera is used with a percentage in weight with respect to the overall preparation comprised between about 1% and about 5%.

12. The preparation as in claim 10, wherein for the production thereof a quantity of Centella Asiatica is used with a percentage in weight with respect to the overall preparation comprised between about 1% and about 5%.

13. The preparation as in claim 10, wherein for the production thereof a quantity of Calendula is used with a percentage in weight with respect to the overall preparation comprised between about 1% and about 5%.

14. The preparation as in claim 10, wherein for the production thereof a quantity of Tea tree oil is used with a percentage in weight with respect to the overall preparation comprised between about 0.05% and about 0.5%.

15. The preparation as in claim 10, wherein for the production thereof a quantity of sweet almond oil is used with a percentage in weight with respect to the overall preparation comprised between about 1% and about 5%.

16. The preparation as in claim 1 for use in treatment of tissue repair of the vaginal mucosa, cervicovaginal atrophy and relative dystrophic conditions, delay in the process of vulvo-vaginal tissue repair after childbirth, in gynecological surgery, in dystrophies that follow chemotherapy treatments or treatments based on ionizing radiations and also in use for auxiliary treatment of the reparatory processes of the anorectal mucosa after proctological surgery, internal and external hemorrhoids, proctitis, cryptitis, anal rhagades, perianal fissures and fistulae.

17. A method for the production of a preparation for vaginal and rectal use comprising hyaluronic acid, the method using micronized hyaluronic acid with an average particle size comprised between about 50 micrometers and about 200 micrometers and with a molecular weight comprised between about 1,000,000 Da and about 1,800,000 Da.
